(19) United States
(12) Patent Application Publication
(10) Pub. No.: US 2010/0125264 A1
(43) Pub. Date: May 20, 2010

(54) Disposable Undergarment with Sanitary Napkin

(76) Inventor: Leatrice A. Naylor, Manassas, VA (US)

Correspondence Address: DIEDERIKS & WHITELAW, PLC, 13885 HEDGEWOOD DR., SUITE 317, WOODBRIDGE, VA, 22193, US

(21) Appl. No.: 12/619,752

(22) Filed: Nov. 17, 2009

Related U.S. Application Data
(60) Provisional application No. 61/115,279, filed on Nov. 17, 2008.

(51) Int. Cl. A61F 13/45; A61F 13/56; A61F 13/68; D05B 97/00

(52) U.S. Cl. 604/385.29; 604/391; 604/396; 112/475.09

(57) ABSTRACT

A disposable undergarment includes a main body portion having inner and outer layers of non-woven viscous material sewn together at outer edge portions, and an absorbent pad sandwiched between the inner and outer layers in a crotch area of the undergarment. Preferably, an antimicrobial coating is applied to the inner and outer layers. The absorbent pad is adhered to the outer layer such that seams do not extend through the absorbent pad. Elastomeric leg bands are sewn to leg portions of the undergarment and first and second elastomeric waist band portions are sewn to waist portions of the undergarment. Adjustable fasteners connect the first and second waist band portions around the waist of a user.

### CROSS-REFERENCE TO RELATED APPLICATIONS

The present invention claims the benefit of U.S. Provisional Patent Application Ser. No. 61/115,279 entitled “Disposable Undergarment with Sanitary Napkin” filed Nov. 17, 2008.

### BACKGROUND OF THE INVENTION

1. Field of the Invention

The present invention pertains to the art of disposable garments and, more particularly, to a disposable absorbent panty.

2. Discussion of the Prior Art

Sanitary napkins are commonly used to absorb menstrual flow and are typically worn between the body of a user and the user's undergarment. One disadvantage with sanitary napkins are their tendency to shift during use, especially when worn during physical activities, which can lead to staining of the undergarment and embarrassing leak-through. Such staining and accidents can be particularly worrisome for young females who are more prone to wearing sanitary napkins over tampons or other products. One proposed solution has been to incorporate sanitary napkins with undergarments. For example, U.S. Pat. No. 5,651,779 teaches a panty liner which is sandwiched between an external layer and an internal moisture permeable layer of the crotch section of a panty. The moisture absorbent layer and moisture impermeable layer are sewn together and sealed with a seam sealer. The panty is waterproof and washable. Alternatively, a removable pad is secured to the panty by hook and loop (e.g., Velcro™) strips. However, users may find the idea of washing absorbent garments undesirable, and securing a pad with various hooks can be time consuming and result in an uncomfortable garment which is more expensive to produce.

Another example, U.S. Patent Application Publication No. 2008/0125737, teaches an undergarment incorporating an integrated sanitary napkin wherein the pad is sewn inside the undergarment. The garment may be disposable or, made of washable materials. Velcro™ straps allow a user to adjust the fit of the undergarment. Although this reference teaches a disposable option, sewing a pad into an undergarment creates undesirable seams through which moisture can migrate.

Additionally, it is known to provide diaper-like disposable undergarments. However, such undergarments are uncomfortable and unattractive. Based on the above, there is seen to be a need in the art for an improved disposable undergarment which is attractive, comfortable, avoids any diaper-like feeling and can be simply manufactured.

### SUMMARY OF THE INVENTION

The present invention is directed to a disposable undergarment incorporating a sanitary pad. The disposable undergarment includes a main body portion having inner and outer layers of non-woven viscous material sewn together at outer edge portions, and an absorbent pad sandwiched between the inner and outer layers in a crotch area of the undergarment. Preferably, an antimicrobial coating is applied to the inner and outer layers. The absorbent pad is adhered to the outer layer such that seams do not extend through the absorbent pad. Elastomeric leg bands are sewn to leg portions of the undergarment and first and second elastomeric waist band portions are sewn to an upper waist portion of the undergarment. Adjustable fasteners connect the first and second waist band portions around the waist of a user.

With this arrangement, the undergarment of the present invention provides a comfortable and attractive option for protection during a menstrual cycle or other occasions when wetness protection is desirable. Additional objects, features and advantages of the present invention will become more readily apparent from the following detailed description of a preferred embodiment when taken in conjunction with the drawings wherein like reference numerals refer to corresponding parts in the several views.

### BRIEF DESCRIPTION OF THE DRAWINGS

FIG. 1 depicts a front view of a fastened disposable undergarment of the present invention; and

FIG. 2 depicts the inside of the disposable undergarment of FIG. 1 in an unfastened configuration.

### DETAILED DESCRIPTION OF THE PREFERRED EMBODIMENT

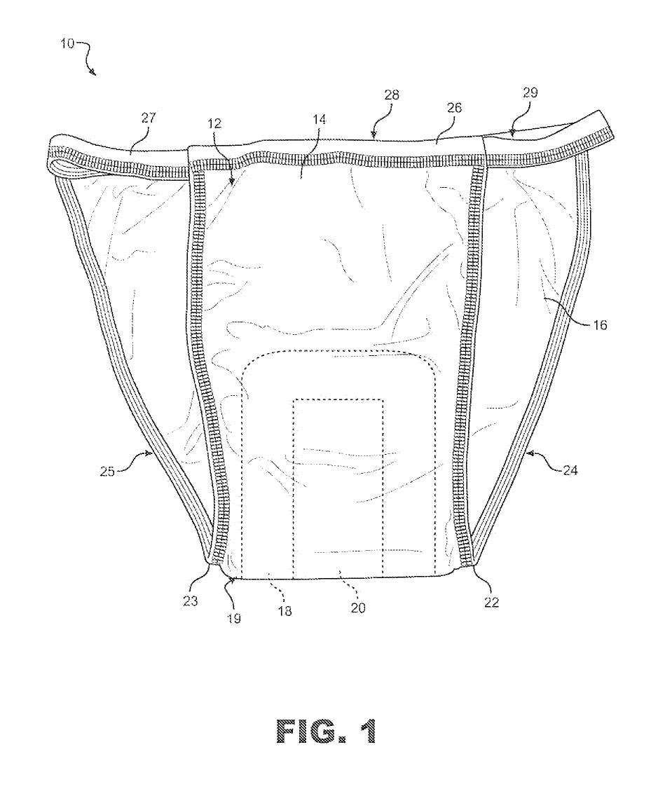

With initial reference to FIG. 1, an improved disposable undergarment 10 is shown including a main body portion 12 comprising an outer material 14 and an inner material 16. An absorbent pad indicated at 18 is sandwiched between outer material 14 and inner material 16 in the crotch area 19 of undergarment 10. Inner and outer materials 14 and 16 are preferably non-woven viscous fabrics sewn together at their outer edges, although other permeable materials known in the art may be utilized. Optionally, impermeable materials or a combination of permeable and impermeable materials may be utilized. In a preferred embodiment, main body portion 12 includes an antimicrobial treatment, such as the one disclosed in U.S. Pat. No. 6,928,664, hereby incorporated by reference.

Absorbent pad 18 is fixed in place with one or more adhesive or bonding strips indicated at 20, which adheres pad 18 to outer material 14. Bonding strips 20 can be in the form of tape strips or may include other types of bonding known in the art such as ultrasonic welding. At this point, it should be noted that pad 18 is not stitched in place and therefore does not include any seams through which moisture can travel. In a preferred embodiment, absorbent pad 18 is a thin ultra-absorbent pad having an impermeable bottom layer and a permeable top layer, although it is contemplated that any number of absorbent pads known in the art may be utilized with the present invention. In addition, it should also be noted that the size of pad 18 can vary in accordance with the invention. For instance, in an alternative embodiment (not shown), absorbent pad 18 can actually take the form of a full panty pad which is sized to fit the entire main body portion 12, which is sandwiched between inner material 16 and outer material 14 and is adhered to outer material 14.

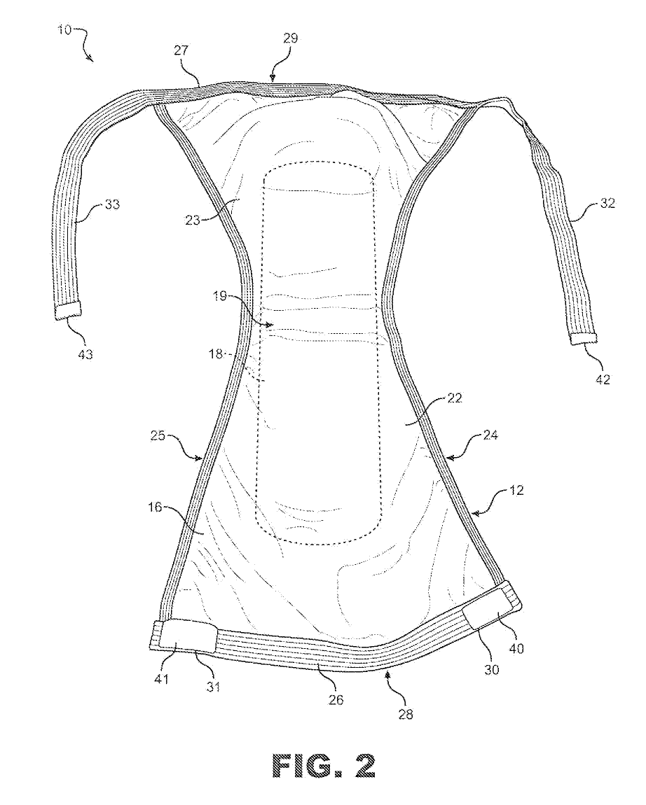

Elastomeric leg bands 22 and 23 are sewn onto outer edges of undergarment 10 at opposing leg portions 24 and 25, and first and second elastomeric waist band portions 26 and 27 are sewn onto outer edges of undergarment 10 at opposing waist portions 28 and 29. In a preferred embodiment, leg bands 22, 23 and waist band portions 26, 27 are sewn using cross-stitching. As best seen in FIG. 2, fasteners are utilized to fasten first and second side portions 30 and 31 of waist portion 28 to respective first and second side portions 32 and 33 of waist portion 29. More specifically, fasteners 40 and 41 located on opposing sides of waist band portion 26 are adapted to fasten to respective fasteners 42 and 43 on opposing sides of waist band portion 27. In the preferred embodiment shown, fasteners 40-43 are in the form of hook and loop (e.g., Velcro™) tabs with fasteners 40 and 41 constituting the hook portions and fasteners 42 and 43 constituting the softer loop portions such that the user's skin will not be irritated by the fasteners. However, other fasteners, such as snap fasteners, may be utilized. This configuration allows the fit of undergarment 10 to be adjusted for user comfort and fit.

Disposable undergarment 10 may be manufactured in a variety of size ranges, styles and colors. Optionally, a number of disposable undergarments 10 sufficient for an average menses cycle may be packaged together. Thus, an attractive, comfortable disposable undergarment 10 is provided for reliable feminine protection which looks like a normal panty and avoids a diaper-like look or feel. Although described with reference to a preferred embodiment of the invention, it should be readily understood that various changes and/or modifications can be made to the invention without departing from the spirit thereof. For instance, although shown in a bikini style panty, it should be understood that the present invention can be provided in any number of desirable styles, including boxers, thongs, briefs, etc.

I claim:

1. A disposable undergarment comprising:
a main body portion including an inner layer of permeable material and an outer layer of permeable material, the inner layer sewn to the outer layer at outer edge portions of the respective inner and outer layers and defining outer seams;
an absorbent pad sandwiched between the inner and outer layers of the main body portion in a crotch area of the main body portion, wherein the outer seams of the undergarment do not extend through the absorbent pad;
elastomeric leg bands sewn to outer edge portions of the inner and outer layers;
a first elastomeric waist band sewn to a first top section of the main body portion, the first elastomeric waist band including first and second opposing side portions having respective first and second fasteners; and
a second elastomeric waist band sewn to a second top section of the main body portion, the second elastomeric waist band including third and fourth opposing side portions having respective third and fourth fasteners adapted to connect in an adjustable fashion to respective first and second fasteners of the first elastomeric waist band.

2. The disposable undergarment of claim 1, wherein at least the outer layer of the main body portion is comprised of a non-woven viscous fabric.

3. The disposable undergarment of claim 1, further comprising:
an antimicrobial coating covering the inner and outer layers.

4. The disposable undergarment of claim 1, further comprising:
adhesive strips adhering the absorbent pad to the outer layer of the main body portion.

5. The disposable undergarment of claim 1, wherein the absorbent pad includes an impermeable bottom layer adjacent the outer layer of the main body portion and a permeable layer adjacent the inner layer of the main body portion.

6. The disposable undergarment of claim 1, wherein the disposable undergarment is a bikini style undergarment.

7. The disposable undergarment of claim 1, wherein the first, second, third and fourth fasteners are in the form of hook and loop-type fasteners.

8. A method of making a disposable undergarment comprising:
providing a main body portion including an inner layer of permeable material and an outer layer of permeable material;
sandwiching an absorbent pad between the inner and outer layers of the main body portion in a crotch area of the main body portion;
sewing the inner layer to the outer layer at outer edge portions of the respective inner and outer layers to define outer seams, wherein the outer seems do not extend through the absorbent pad;
sewing elastomeric leg bands to outer edge portions of the inner and outer layers;
sewing a first elastomeric waist band to a first top section of the main body portion, the first elastomeric waist band including first and second opposing side portions having respective first and second fasteners; and
sewing a second elastomeric waist band to a second top section of the main body portion, the second elastomeric waist band including third and fourth opposing side portions having respective third and fourth fasteners adapted to connect in an adjustable fashion to respective first and second fasteners of the first elastomeric waist band.

9. The method of claim 8, wherein at least the outer layer of the main body portion is comprised of a non-woven viscous fabric.

10. The method of claim 8, further comprising:
coating the inner and outer layers with an antimicrobial coating.

11. The method of claim 8, further comprising:
adhering the absorbent pad to the outer layer of the main body portion.

12. The method of claim 8, wherein the absorbent pad includes an impermeable bottom layer adjacent the outer layer of the main body portion and a permeable layer adjacent the inner layer of the main body portion.

13. The method of claim 8, wherein the disposable undergarment is a bikini style undergarment.

14. The method of claim 8, wherein the first, second, third and fourth fasteners are in the form of hook and loop-type fasteners.
